(19) United States
(12) Patent Application Publication
(10) Pub. No.: US 2008/0146914 A1
(43) Pub. Date: Jun. 19, 2008

(54) SYSTEM, METHOD AND APPARATUS FOR CANCER IMAGING

(75) Inventors: Jason Andrew Polzin, Lake Mills, WI (US); Ralph Eugene Hurd, Milpitas, CA (US); Alexander Ganin, Whitefish Bay, WI (US); Hari Hariharan, Pewaukee, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/612,727

(22) Filed: Dec. 19, 2006

(51) Int. Cl. A61B 5/055

(52) U.S. Cl. 600/420

(57) ABSTRACT

A method for imaging cancer using a combined PET-MRI system takes advantage of the performance characteristics for both PET and MRI in the context of cancer imaging. MRI is used to assess a large area of the body with high sensitivity for cancer and PET is then used in localized areas of concern to provide physiological information. Optionally, MRI may also then be used to re-scan the localized areas of concern with high spatial resolution and additional tissue contrasts to provide anatomical information and soft tissue contrast to supplement the PET information. The use of a combined PET-MRI system ensures that the imaging data from both modalities is accurately referenced to the same locations in the body.

### TECHNICAL FIELD

The present invention relates generally to positron emission tomography (PET) and magnetic resonance imaging (MRI), and more specifically, to a method and apparatus for imaging cancer using a combined PET-MRI system.

### BACKGROUND

PET imaging involves the creation of tomographic images of positron emitting radionuclides in a subject of interest. A radionuclide-labeled pharmaceutical, or “radiopharmaceutical”, is administered to an imaging subject. The subject is positioned within a PET imaging system comprising a detector ring and detection electronics. As the radionuclides decay, positively charged photons known as “positrons” are emitted. For commonly used radiopharmaceuticals such as FDG, (i.e., ¹⁸F-fluorodeoxyglucose), these positrons travel only a few millimeters through the tissues of the subject before colliding with an electron, resulting in mutual annihilation. The positron/electron annihilation results in a pair of oppositely-directed gamma rays that are emitted with approximately 511 keV energy.

It is these gamma rays that are detected by the scintillators of the detector ring. When struck by a gamma ray, the scintillating material in these components emits light, which is detected by a photodetector component, such as a photodiode or photomultiplier tube. The signals from the photodetectors are processed as incidences of gamma rays. When two gamma rays strike oppositely positioned scintillators at approximately the same time, a coincidence is registered. Data sorting units process the coincidences to determine which are true coincidence events and sort out data representing dead times and single gamma ray detections. The coincidence events are binned and integrated to form frames of PET data which may be reconstructed as images depicting the distribution of the radionuclide-labeled pharmaceutical in the subject.

MRI is a medical imaging modality that can create pictures of the inside of a human body without using x-rays or other ionizing radiation. MRI uses a powerful magnet to create a strong, uniform, static magnetic field (i.e., the “main magnetic field”). When a human body, or part of a human body, is placed in the main magnetic field, the nuclear spins that are associated with the hydrogen nuclei in tissue water become polarized. This means that the magnetic moments that are associated with these spins become preferentially aligned along the direction of the main magnetic field, resulting in a small net tissue magnetization along that axis (the “z axis”, by convention). An MRI system also comprises components called gradient coils that produce smaller amplitude, spatially varying magnetic fields when current is applied to them. Typically, gradient coils are designed to produce a magnetic field component that is aligned along the z axis, and that varies linearly in amplitude with position along one of the x, y or z axes. The effect of a gradient coil is to create a small ramp on the magnetic field strength, and concomitantly on the resonant frequency of the nuclear spins, along a single axis. Three gradient coils with orthogonal axes are used to “spatially encode” the MR signal by creating a signature resonance frequency at each location in the body. Radio frequency (RF) coils are used to create pulses of RF energy at or near the resonance frequency of the hydrogen nuclei. These coils are used to add energy to the nuclear spin system in a controlled fashion. As the nuclear spins then relax back to their rest energy state, they give up energy in the form of an RF signal. This signal is detected by the MRI system, and combined with multiple additional such signals may be used to reconstruct an MR image using a computer and known algorithms.

Both PET and MRI are used routinely in cancer detection and diagnosis. PET imaging in the clinical setting is most commonly performed using a glucose analogue, ¹⁸F-fluorodeoxyglucose (¹⁸F-FDG), to measure cellular metabolism. ¹⁸F-FDG is taken up by cells and phosphorylated in parallel to glucose. The amount of ¹⁸F-FDG uptake by a cell is a measure of the amount of glucose metabolism it is performing. Most cancer cells have increased metabolic activity and uptake ¹⁸F-FDG at an increased rate relative to normal cells. A focal area of increased ¹⁸F-FDG uptake is therefore considered a marker for malignancy. The use of MRI in cancer imaging relies primarily on detecting gross anatomical changes or changes in microvascular anatomy or physiology that are associated with the presence of a growing malignancy. In the context of cancer detection and diagnosis, several MRI techniques are known that allow sensitive detection of cancerous lesions throughout the body. For example, a Short Tau Inversion Recovery (STIR) weighted sequence may be used, wherein metastatic lesions appear as focal bright areas on a dark background. Alternatively, a MR contrast agent may be used to create image contrast between a cancerous lesion and background tissue. A MR contrast agent may be administered intravenously in an imaging subject prior to MR imaging. For example, a paramagnetic contrast agent may be used that pools in areas of high capillary density and leaky capillary walls, such as are typical of rapidly growing tissues, and may be detected as a high MRI signal on a T1-weighted image. Cancer cells stimulate increased vascular density and increased vascular permeability in their environment through the release of angiogenesis factors. Detection of a focal area of increased signal intensity on a T1-weighted image with contrast agent is a sensitive indicator of cancer.

The role of imaging in cancer includes the detection and diagnosis of a primary tumor and subsequent staging to determine the extent of metastatic spread of the disease to other locations in the body. For the staging application, a whole-body imaging approach is desirable, however, whole-body imaging requires the ability to image a large region with high spatial resolution and high detection sensitivity in a reasonable scan time. Whole-body PET imaging requires impracticably long scan times. For example, imaging a torso using PET may require a half hour of scanning and multiple additional imaging stations would be required to achieve whole-body coverage. In addition, the practical spatial resolution achievable for a whole-body scan with current PET systems is 5-10 mm, resulting in unacceptable sensitivity to small cancers. However, ¹⁸F-FDG uptake in cancers correlates with histological type and clinically aggressive behavior and as such is an important tool in cancer diagnosis and management. Using currently available technologies, a whole-body MRI exam with sub-millimeter resolution, may be completed in less than 20 min. Whole-body MRI has demonstrated very high sensitivity to cancer. In addition, MRI provides anatomical imaging and soft-tissue contrast. Use of a combined PET-MRI system yields accurate co-registration of the anatomical information provided by MRI with the metabolic information provided by PET. Accordingly, it would be desirable to provide an integrated PET-MR system and method for cancer imaging that takes advantage of the unique strengths of both imaging modalities.

### BRIEF DESCRIPTION OF THE INVENTION

In accordance with an embodiment, a method for cancer imaging in a PET-MRI system includes acquiring magnetic resonance (MR) images of a first region in an imaging subject using a MR imaging protocol, the MR images having characteristics, defining a second region of the imaging subject based on at least the characteristics of the MR images, the second region being a sub-region of the first region, and acquiring positron emission tomography (PET) images of the second region

In accordance with another embodiment, a computer-readable medium having computer-executable instructions for performing a method for cancer imaging in a PET-MRI system includes program code for acquiring magnetic resonance (MR) images of a first region in an imaging subject using a MR imaging protocol, the MR images having characteristics, program code for defining a second region of the imaging subject based on at least the characteristics of the MR images, the second region being a sub-region of the first region, and program code for acquiring positron emission tomography (PET) images of the second region.

In accordance with another embodiment, a combined PET-MRI system includes a positron emission tomography (PET) imaging assembly having a detector positioned to detect PET emissions from an imaging subject and a coincidence processor coupled to receive output from the detector, a magnetic resonance (MR) imaging assembly comprising a magnet, a plurality of gradient coils, a radio frequency coil, a radio frequency transceiver system and a pulse generator module, and a processor coupled to the PET imaging assembly and the MR imaging assembly and configured to acquire MR images of a first region in the imaging subject using a MR imaging protocol and the MR imaging assembly, the MR images having characteristics, to define a second region of the imaging subject based on at least the characteristics of the MR images, the second region being a sub-region of the first region, and to acquire PET images of the second region using the PET assembly.

### BRIEF DESCRIPTION OF THE DRAWINGS

Embodiments are illustrated by way of example and not limitation in the accompanying drawings, in which like reference numerals indicate corresponding, analogous or similar elements, and in which:

FIG. 1 is a schematic block diagram of a combined PET-MRI system in accordance with an embodiment;

FIG. 2 is a flowchart showing a method for cancer imaging in a combined PET-MRI system in accordance with an embodiment; and

FIG. 3 is a flowchart showing a method for cancer imaging in a combined PET-MRI system in accordance with an alternative embodiment.

### DETAILED DESCRIPTION

In the following detailed description, numerous specific details are set forth in order to provide a thorough understanding of embodiments. However it will be understood by those of ordinary skill in the art that the embodiments may be practiced without these specific details. In other instances, well-known methods, procedures, components and circuits have not been described in detail so as not to obscure the embodiments.

A combined PET-MRI system may be used for cancer imaging to take advantage of performance characteristics for both PET and MRI in the context of cancer imaging to increase overall imaging effectiveness. MRI may first be used to assess a large area of the body using MR imaging protocols that have high detection sensitivity for cancer. After identifying localized areas of concern on the MR images, PET may then be used to scan a more limited volume encompassing the areas of concern. PET provides metabolic information about the tissue in these smaller regions. Optionally, MRI may be used to re-scan these localized areas of concern with high spatial resolution and additional tissue contrasts to provide high-resolution anatomical information complementary to the metabolic PET information. The use of a combined PET-MRI system ensures that the imaging data from both modalities is accurately referenced to the same locations in the body.

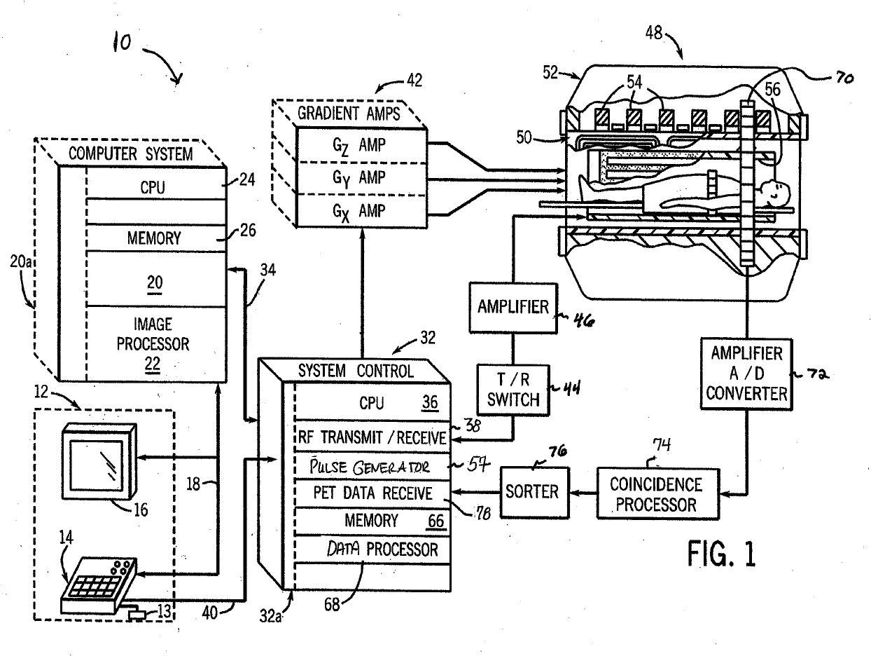

Referring to FIG. 1, the major components of an exemplary combined PET-MR imaging system 10 that may incorporate embodiments of the present invention are shown. The operation of the system may be controlled from an operator console 12 which includes a keyboard or other input device 13, a control panel 14, and a display screen 16. The console 12 communicates through a link 18 with a separate computer system 20 that enables an operator to control the production and display of images on the display screen 16. The computer system 20 includes a number of modules which communicate with each other through electrical and/or data connections, for example, such as are provided by using a backplane 20a. Data connections may be direct wired links or may be fiber optic connections or wireless communications links or the like. The modules include an image processor module 22, a CPU module 24 and a memory module 26, which may include a frame buffer for storing image data arrays. In an alternative embodiment, the image processor module 22 may be replaced by image processing functionality on the CPU module 24. The computer system 20 may also be connected to permanent or back-up memory storage, a network, or may communicate with a separate system control 32 through a link 34. The input device 13 can include a mouse, joystick, keyboard, track ball, touch activated screen, light wand, voice control, or any similar or equivalent input device, and may be used for interactive geometry prescription.

The system control 32 includes a set of modules in communication with each other via electrical and/or data connections 32a. Data connections 32a may be direct wired links or may be fiber optic connections or wireless communication links or the like. System control 32 is connected to the operator console 12 through a communications link 40. It is through link 40 that the system control 32 receives commands from the operator to indicate the scan sequence or sequences that are to be performed. The modules of system control computer 32 include a CPU module 36 and a pulse generator module 57 that connects to the operator console 12 through a communications link 40. For MR data acquisition, an RF transmit/receive module 38 commands the scanner 48 to carry out the desired scan sequence, by sending instructions, commands, and/or requests describing the timing, strength and shape of the RF pulses and pulse sequences to be produced, to correspond to the timing and length of the data acquisition window. In this regard, a transmit/receive switch 44 controls the flow of data via amplifier 46 to scanner 48 from RF transmit module 38 and from scanner 48 to RF receive module 38. The system control 32 also connects to a set of gradient amplifiers 42 to indicate the timing and shape of the gradient pulses that are produced during the scan.

The gradient waveform instructions produced by system control 32 are sent to the gradient amplifier system 42 having Gx, Gy, and Gz amplifiers. Gradient amplifiers 42 may be external of scanner 48 or system control 32, or may be integrated therein. Each gradient amplifier excites a corresponding physical gradient coil in a gradient coil assembly generally designated 50 to produce the magnetic field gradients used for spatially encoding acquired signals. The gradient coil assembly 50 forms part of a magnet assembly 52 which includes a polarizing magnet 54 and a RF coil assembly 56. Alternatively, the gradient coils of gradient coil assembly 50 may be independent of magnet assembly 52. RF coil assembly 56 may include a whole-body RF transmit coil as shown, surface or parallel imaging coils (not shown), or a combination of both. The RF coils of the RF coil assembly 56 may be configured for both transmitting and receiving, or for transmit-only or receive-only. A pulse generator 57 may be integrated into system control 32 as shown, or may be integrated into the scanner equipment 48 and produces pulse sequences or pulse sequence signals for the gradient amplifiers 42 and/or the RF coil assembly 56. Alternatively, RF coil assembly 56 may be replaced or augmented with surface and/or parallel transmit coils. The MR signals resulting from the excitation pulses, emitted by the excited nuclei in the patient, may be sensed by the whole body coil or by separate receive coils, such as parallel coils or surface coils, and are then to the RF transmit/receive module 38 via T/R switch 44. The MR signals are demodulated, filtered, and digitized in the data processing section 68 of the system control 32.

An MR scan is complete when one or more sets of raw k-space data has been acquired in the data processor 68. This raw k-space data is reconstructed in data processor 68 which operates to transform the data (through Fourier transformation or other technique) into image data. This image data is conveyed through the link 34 to the computer system 20 where it is stored in memory 26. Alternatively, in some systems, computer system 20 may assume the image reconstruction or other functions of data processor 68. In response to commands received from the operator console 12, the image data stored in memory 26 may be archived in long term storage or may be further processed by the image processor 22 and conveyed to the operator console 12 and presented on the display 16.

In the combined PET-MRI system 10, scanner 48 also contains a positron emission detector 70, configured to detect gamma rays from positron annihilations emitted from a subject. Detector 70 preferably includes a plurality of scintillators and photodetectors arranged about a gantry. Detector 70 may, however, be of any suitable construction for acquiring PET data. In addition, the scintillator components, photodetectors, and other electronics of the detector 70 need not be shielded from the magnetic fields and/or RF fields applied by the MR components 54, 56. However, it is contemplated that embodiments of the present invention may include such shielding as known in the art, or may be combined with various shielding techniques.

Gamma ray incidences detected by detector 70 are transformed, by the photodetectors of the detector 70, into electrical signals and are conditioned by a series of front-end electronics 72. These conditioning circuits 72 may include various amplifiers, filters, and analog-to-digital converters. The digital signals output by front end electronics 72 are then processed by a coincidence processor 74 to match gamma ray detections as potential coincidence events. When two gamma rays strike detectors approximately opposite one another, it is possible, absent the interactions of random noise and signal gamma ray detections, that a positron annihilation took place somewhere along the line between the detectors. Thus, the coincidences determined by coincidence processor 74 are sorted into true coincidence events and are ultimately integrated by data sorter 76. The coincidence event data, or PET data, from sorter 76 is received by the system control 32 at a PET data receive port 78 and stored in memory 66 for subsequent processing by processor 68. PET images may then be reconstructed by image processor 22 and may be combined with MR images to produce hybrid structural and metabolic or functional images. Conditioning circuits 72, coincidence processor 74 and sorter 76 may each be external of scanner 48 or control system 32 or may be integrated therein.

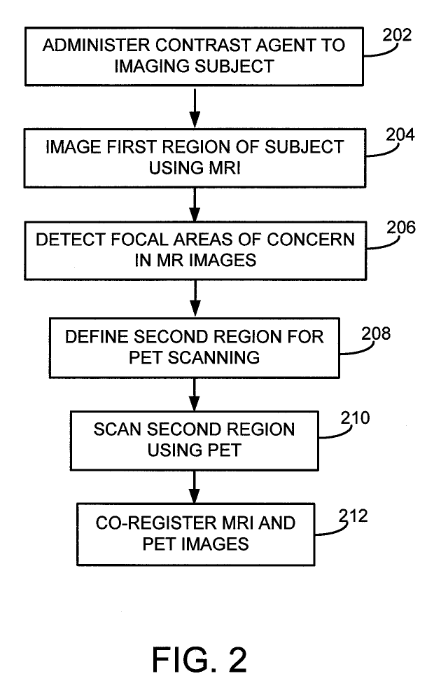

FIG. 2 is a flowchart showing a method for cancer imaging in a combined PET-MRI system in accordance with an embodiment. At block 202, a contrast agent is administered to the imaging subject. The contrast agent may be a pharmaceutical that provides contrast for a PET exam. A pharmaceutical that provides contrast for a MRI exam may also be administered. For example, a PET radiopharmaceutical, such as ¹⁸F-FDG may be administered singly, or mixed together with a MRI contrast agent, such as Gadolinium diethylenetriamine penta-acetic acid (Gd-DTPA) and delivered to the subject as a single intravenous injection. The typical injected activity of ¹⁸F-FDG depends on patient size and body weight (e.g. size of 175 cm and body weight of 75 kg may typically receive 440 MBq injected activity). A typical dose of Gd-DTPA is 0.1-0.2 mmol Gd-DTPA per kilogram of imaging subject body weight. Alternatively, a PET radiopharmaceutical and a MRI contrast agent may be administered as separate intravenous injections prior to scanning. The PET radiopharmaceutical and the MRI contrast agent may be injected separately, but in rapid succession, such that they are effectively delivered at approximately the same time and are mixed in the bloodstream. Preferably, the contrast agent or agents are administered within a short timeframe prior to scanning with the PET-MRI system. In another embodiment, a radionuclide-labeled MRI contrast agent may be administered that provides contrast for both PET and MRI. Alternatively, other types of MR contrast agents, such as superparamagnetic iron oxide particles, may be used.

At block 204, a first region of the imaging subject is imaged or scanned using MRI to acquire image(s). For example, the first region may be the entire imaging subject, where a “whole-body” MRI protocol is prescribed to scan the patient from head to toe. In another example, the first region may be a volume encompassing both breasts of a patient wherein a bilateral breast protocol is prescribed. The MRI protocol may be prescribed by, for example, a system operator via an operator console 12 (shown in FIG. 1). MRI techniques that are sensitive to cancer may be included in the MRI protocol. For example, T2-weighted imaging, such as is provided with a Short Tau Inversion-Recovery (STIR) sequence may be used for lesion detection. Alternatively, or additionally, T1-weighted imaging may be used to detect focal areas of paramagnetic contrast agent accumulation. Alternatively, or additionally, out-of-phase gradient-recalled echo imaging may be used to look for foci of cancer. Other MRI techniques known in the art and not described herein may be used for the MRI exam. Parallel imaging techniques and phased array coils may be used to speed up the imaging as known in the art.

At block 206, the MR image(s) acquired from the MRI exam at block 204 may be examined or inspected to identify or detect focal areas of concern within the first region. An area of concern (or suspect area) may be identified based on characteristics of the MR images, for example, as bright areas or areas of contrast enhancement on at least one of the acquired MR images. For example, an area of concern or suspect area may be a bright area on a STIR-weighted image or a bright area on a T1-weighted image acquired after administration of a paramagnetic contrast agent. A suspect area or area of concern may be, for example, a location in the first region that is a suspected cancerous lesion. Areas of concern may be identified by visual inspection of the images, or through use of a computer-aided detection tool based on automated lesion discrimination techniques. The PET-MRI system may include image manipulation and image processing tools that aid in visual inspection of the images, such as a tool to zoom into a suspicious region and graphically prescribe a volume that may be used to define either additional MRI or PET imaging, or both.

At block 208, a second region of the imaging subject is for scanning using PET. The second region is a sub-region of the first region. The second region may be defined to include, for example, any suspect areas identified at block 206 or any areas of contrast enhancement in the MR images. For example, for a bone cancer application, the second region may be defined to include locations of possible enhancing lesions identified on images from a whole-body MR imaging. In another example, in a breast cancer detection application, the second region may be defined to include suspect lesions detected by the MR imaging. In addition, the second region may be defined based on other criteria such as prior identification of a region suspect for the presence of cancer using, for example, another imaging modality, or a non-imaging clinical examination. The second region may be defined automatically by the PET-MRI system using image processing techniques known in the art or may be defined by a system operator via an operator console 12 (shown in FIG. 1). The high sensitivity of MRI to cancer allows for reasonable exclusion of further imaging in certain regions, and for targeting certain regions using PET.

At block 210, the second region of the imaging subject is scanned using a PET exam to acquire PET image(s). As mentioned, the second region scanned using PET is a sub-region of the first region scanned using MRI. As a result, the total volume to be scanned using PET may be reduced relative to the size of the first region. Accordingly, the PET images may be obtained either with reduced total acquisition time or with much higher spatial resolution than would be practical for a whole-body or other large volume PET exam.

At block 212, the PET images acquired at block 210 and the MRI images acquired at block 204 may be co-registered to facilitate comparison and evaluation. Alternatively, the PET images and MRI images may be “fused” to form composite images for evaluation, i.e., the information from a PET image and the information from a MRI image from the same location may be combined into a single image. For example, metabolic information from PET may be displayed as a semi-transparent color overlay on a gray-scale anatomical MR image.

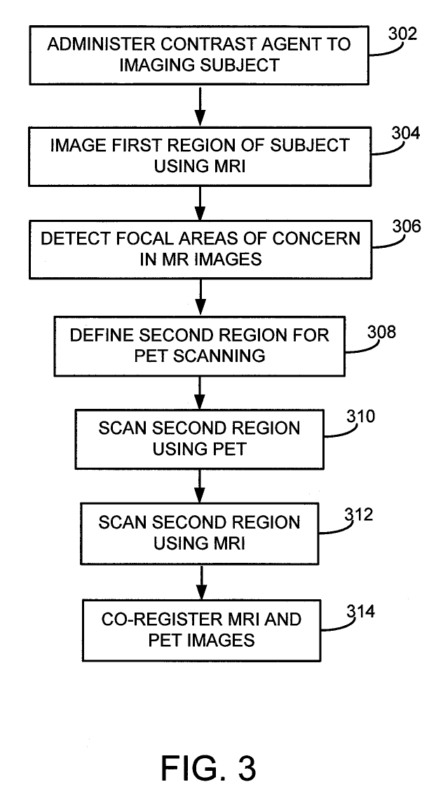

FIG. 3 is a flowchart showing a method for cancer imaging in a combined PET-MRI system in accordance with an alternative embodiment. The process of blocks 302-310 are similar to that described above with respect to blocks 202-210 of FIG. 2. At block 302, a contrast agent, e.g., a PET pharmaceutical, is administered to an imaging subject. At block 304, a first region of the imaging subject is imaged or scanned using a MRI exam to acquire MR image(s). At block 306, the MR image(s) acquired from the MRI exam at block 304 may be examined or inspected to identify or detect focal areas of concern within the first region. At block 308, a second region of the imaging subject is defined that is to be scanned using a PET exam. The second region is a sub-region of the first region. At block 310, the second region of the imaging subject is scanned using a PET exam to acquire PET image(s).

At block 312, a second MRI exam may be performed, re-scanning the second region that was scanned using a PET exam at block 310. Accordingly, the MRI exam of the second region may be used to obtain higher spatial resolutions and/or to acquire additional image contrasts. The soft tissue contrast and anatomical detail that may be obtained using MRI supplements the metabolic information from the PET images and may allow, for example, for more confident identification of areas of increased but normal radiopharmaceutical uptake on the PET images. In an alternative embodiment, the PET scanning of the second region at block 310 and the MRI scanning of the second region at block 312 may be performed simultaneously. At block 314, the PET images acquired at block 310 and the MR images acquired at block 304 and/or the MR images acquired at block 312 may be co-registered and/or fused to facilitate comparison and evaluation.

Computer-executable instructions for imaging cancer using a PET-MRI system according to the above-described method may be stored on a form of computer readable media. Computer readable media includes volatile and nonvolatile, removable and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital versatile disks (DVD) or other optical storage, magnetic cassettes, magnetic tape, magnetic disk storage or other magnetic storage devices, or any other medium which can be used to store the desired instructions and which may be accessed by the PET-MRI system 10 (shown in FIG. 1), including by internet or other computer network forms of access.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. The order and sequence of any process or method steps may be varied or re-sequenced according to alternative embodiments.

Many other changes and modifications may be made to the present invention without departing from the spirit thereof. The scope of these and other changes will become apparent from the appended claims.

What is claimed is:

1. A method for cancer imaging in a PET-MRI system comprising:
acquiring magnetic resonance (MR) images of a first region in an imaging subject using a MR imaging protocol, the MR images having characteristics;
defining a second region of the imaging subject based on at least the characteristics of the MR images, the second region being a sub-region of the first region; and
acquiring positron emission tomography (PET) images of the second region.

2. A method according to claim 1, further comprising acquiring additional MR images of the second region.

3. A method according to claim 1, further comprising co-registering at least one MR image with at least one PET image.

4. A method according to claim 1, further comprising fusing at least one MR image with at least one PET image.

5. A method according to claim 1, further comprising, before acquiring MR images of the first region, administering a contrast agent to the imaging subject.

6. A method according to claim 5, wherein the contrast agent is a PET contrast agent.

7. A method according to claim 5, wherein the contrast agent comprises a PET contrast agent combined with a MR imaging contrast agent.

8. A method according to claim 5, wherein the contrast agent comprises a radionuclide-labeled MR imaging contrast agent.

9. A method according to claim 1, wherein the MR imaging protocol comprises whole-body scanning.

10. A method according to claim 1, wherein the MR imaging protocol comprises short tau inversion recovery scanning.

11. A method according to claim 1, wherein the MR imaging protocol comprises out-of-phase gradient-recalled echo scanning.

12. A method according to claim 1, wherein the MR imaging protocol comprises T1-weighted scanning.

13. A method according to claim 1, wherein defining a second region of the imaging subject comprises:
detecting at least one suspect area for the presence of cancer in at least one of the MR images; and
defining the second region to include the at least one suspect area.

14. A method according to claim 13, wherein the at least one suspect area is detected based on at least the characteristics of the MR images.

15. A computer-readable medium having computer-executable instructions for performing a method for cancer imaging using a PET-MRI system, the computer-readable medium comprising:
program code for acquiring magnetic resonance (MR) images of a first region in an imaging subject using a MR imaging protocol, the MR images having characteristics;
program code for defining a second region of the imaging subject based on at least the characteristics of the MR images, the second region being a sub-region of the first region; and
program code for acquiring positron emission tomography (PET) images of the second region.

16. A computer readable medium according to claim 15, further comprising program code for acquiring MR images of the second region.

17. A combined PET-MRI system comprising:
a positron emission tomography (PET) imaging assembly comprising a detector positioned to detect PET emissions from an imaging subject and a coincidence processor coupled to receive output from the detector;
a magnetic resonance (MR) imaging assembly comprising a magnet, a plurality of gradient coils, a radio frequency coil, a radio frequency transceiver system, and a pulse generator module; and
a processor coupled to the PET imaging assembly and the MR imaging assembly and configured to:
acquire MR images of a first region in the imaging subject using a MR imaging protocol and the MR imaging assembly, the MR images having characteristics;
define a second region of the imaging subject based on at least the characteristics of the MR images, the second region being a sub-region of the first region; and
acquire PET images of the second region using the PET imaging assembly.

18. A combined PET-MRI system according to claim 17, wherein the processor is further configured to acquire MR images of the second region using an MR imaging protocol and the MR imaging assembly.

19. A combined PET-MRI system according to claim 17, wherein the processor is further configured to co-register at least one MR image with at least one PET image.

20. A combined PET-MRI system according to claim 17, wherein the processor is further configured to fuse at least one MR image with at least one PET image.
